(19) United States
(12) Patent Application Publication
(10) Pub. No.: US 2007/0009607 A1
(43) Pub. Date: Jan. 11, 2007

(54) Antibacterial/anti-infalmmatory composition and method

(76) Inventor: George Jones, Bonita Springs, FL (US)

Correspondence Address: EDWARD M. LIVINGSTON, PA, 963 TRAIL TERRACE DRIVE, NAPLES, FL, 34103, US

(21) Appl. No.: 11/178,893

(22) Filed: Jul. 11, 2005

(51) Int. Cl. A61K 31/573; A61K 31/22; A61K 31/192; A61K 35/12; A61M 31/00

(52) U.S. Cl. 424/522; 514/171; 514/568; 604/500; 514/547

(57) ABSTRACT

An antibacterial/anti-inflammatory composition and method wherein the pairing of synergistic active ingredients with a natural enhancer for a more efficacious formulation for topical application treatments is used. Active ingredients hydrocortisone 1% and benzoyl peroxide 10% are paired with Emu Oil to increase the percentage of absorption of the ingredients through the skin. The preferred method of use of the composition is via a dual-pharmaceutical delivery syringe that isolates components (1) having a barrel (9), a piston (8) and a commonly shared split nozzle (5). The barrel (9) has two syringe compartments (2) and (14) attached to one another and connected to the split nozzle (5). The piston (8) has two plungers (6) connected via a bridge (12). A user applies force to the piston (8), dispersing the substances in the syringe compartments (2) and (14) through the respective sides of the split nozzle (5) onto the user's skin. The user then massages the substances into the skin for “hands-on healing.”

### BACKGROUND OF THE INVENTION

This invention relates to antibacterial/anti-inflammatory compositions, more specifically, an improved antibacterial/anti-inflammatory composition comprising the pairing of two synergistic dissimilar active ingredients with an enhancing agent, Emu Oil, for use in acne, rosacea, minor skin infections and wounds and its method of use. The antibacterial provides the primary pharmaceutical mode of action of the composition assisted by the synergistic activity of the anti-inflammatory and Emu Oil for an enhanced composition for improved use for skin afflictions acne, rosacea, minor skin infections and wounds.

The term “composition” as defined is the act of combining to form a whole. The method of use of the present invention is the act of contiguously delivering the paired active ingredients beyond the physical delivery system whereas admixture takes place at the discretion of the consumer on site of affliction.

I. Common Skin Ailments

A. Acne

It is estimated that about 45 million Americans have acne vulgaris, a chronic disease involving the pilosebaceous follicles which affects both adults and adolescents alike. Approximately 90% of all teens are affected by acne to some extent. The impact of acne may appear minimal to an observer, but may be significant to the young person involved.

Sebaceous glands are found most abundantly on the face and scalp, though they are present on every part of the skin except the palms of the hands and soles of the feet. Though the sebaceous gland is a mini organ, it is anatomically and functionally related to the hair follicle. Cutaneous disorders attributed to the sebaceous gland are really disorders of the entire pilosebaceous unit. The areas most commonly involved in acne are the face, upper chests, and back. Other less common areas include the upper arms, buttocks, and upper thighs.

Acne arises from the interaction of four factors: (1) excess sebum production caused by androgenic stimulation of sebaceous glands; (2) outlet obstruction of the sebaceous follicle arising from excess production of keratinocytes (the basic cell of the epidermis); (3) increased proliferation of the bacteria Propionibacterium acne that normally live in the sebaceous follicle; and (4) inflammation caused by sebum escaping into the surrounding skin.

Obstruction of the sebaceous follicle is the primary pathologic event in acne, giving rise to the micro-comedo, the precursor of all acne lesions. Once the follicle is plugged, the lower portion of the follicle becomes engorged and distended with sebaceous discharge and keratinocytes. While the pore remains closed, the lesion is called a closed comedo, commonly known as a whitehead. The closed comedo is 1-3 mm in diameter, white or flesh-colored, and very slightly raised.

When the follicle enlarges enough to stretch the pore and the trapped matter is exposed to air, oxidization occurs, causing the characteristic dark appearance of open comedones or blackheads. Open comedones are flat or slightly raised, brown-to black papules about 3-5 mm in diameter.

Early acne, involving a majority of open and closed comedones, is a noninflammatory process. As dilation of the follicle continues, the follicular epithelium is disrupted and irritants such as sebum, hair and keratinocytes are released into the surrounding dermis. This leakage causes an inflammatory reaction and initiates the formation of the inflammatory lesion papules, pustules, and nodules.

Although P. acnes is a live bacterium living in the follicle, it dies when the follicular structure is disrupted. Toxins are released into the dermis, which increases inflammation. Therefore, uncomplicated, inflammatory acne is actually a sterile process and not a skin infection.

As inflammation continues to worsen, larger papules and pustules are created. Acne papules are pink or red and 2-5 mm in diameter. Pustules contain grossly purulent material. Acne nodules are solid, raised inflammatory lesions that exceed 6-10 mm in diameter and are situated deeper in the dermis. The acne cyst is a large nodule that has suppurated and become fluctuant. Scars form as a result of damage to the surrounding dermis and may appear as small deep punched out pits (“ice picks”), atrophic macules, hypertrophic papules, or broad, sloping depressions. The treatment of acne is aimed at preventing scars from forming.

B. Rosacea

Rosacea, formally known as acne rosacea, is a chronic inflammatory eruption of the nose and adjoining flush areas of the face. Rosacea is characterized by erythema, papules, pustules, telangiectasia and frequently by hypertrophy of the sebaceous glands. The skin disorder occurs most often in women between the ages of thirty and fifty. In its mild form, rosacea brings about a slight flushing of the nose and cheeks and, in some cases, the chin and forehead. However, serious cases have been observed in men where deep red or purple lesions appear and include a chronic dilation of the superficial capillaries (telangiectasia). It is in this serious form that inflammatory acneiform pustules are present on the face, perhaps affecting the eyes or eyelids.

Another acute form of rosacea is known as granulomatous rosacea. It is considered to be a distinct form of the papular aspect of the disease wherein discreet pustules appear as yellowish brown nodules and as epithelioid cell granulomatous.

Although the etiology of rosacea is not fully known, several contributing factors have been suggested, including the disease being endocrine-based, that is, based from hormones; vasomotor liability, that is, related to menopause; and due to increased skin temperature.

C. Skin Infections

The skin is the largest organ of the human body. It acts as a barrier to an ingress of pathogens and toxic chemicals and an egress of physiologic fluids. The skin can be breached by many different means, whether from an accident, intentionally, such as in surgical incision cases, a situational condition such as a pressure bed sore or biological/hereditary conditions such as acne and rosacea. The skin of healthy individuals normally repairs itself. However, in millions of others, complications may arise due to the failure of the skin to repair itself, such as secondary infections along with swelling and inflammation.

In 2002, there were more than 33 million office visits to dermatologists in the United States, many due to open skin lesions. Breach of the skin barrier, micro vascular insufficiency and secondary bacterial invasion may lead to increased risk of skin ulceration, which could possibly lead to amputation.

Decubitus ulcers, commonly called pressure sores or bed sores, have three to four million sufferers per year. Management of patients with chronic wounds can be resource-intensive in terms of costs for supplies and medications, as well as in terms of facility and nursing time charges. In fact, the U.S. spends billions of dollars yearly in the management of pressure ulcers in the acute care setting alone. Estimates of average costs per episode of care for the major chronic wound types (pressure, venous and diabetic ulcers) range from 10 to 30 thousand dollars.

While primary damage may be inflicted by different causes, the healing of skin ulcers, traumatic wounds, burns and other open skin wounds and injuries usually undergo a sequence of steps that results in restoration of structural integrity of the skin. These steps include: 1) wound cleansing, which is achieved through the influx of an inflammatory cell; 2) proliferation of stromal cells to initiate the restorative processes; and 3) ingrowth of blood vessels to provide nutritional support for the regeneration. Effective treatment may be expected to promote wound healing through the support of these steps and protection of the area of open injury against bacterial infection and dehydration.

The natural environment of a cell is a moist one. Indeed, moisture or water is vital to life and most of the body's cells require an aqueous environment to live and function. Thus, dry cells are usually dead cells. For example, cells of the outer epidermis or stratum corneum, such as the hair and the nails, are incapable of reproducing except at their point of origin (the basal layer, the follicle and the nail matrix, respectively), which is in a moist environment, and their role is usually protective. The keratinocyte cells of the stratum corneum originate in the moist basal layer of the epithelium. As the cells travel upwards, they lose their nuclei and fill the tough protein keratin to become equipped for their role as the barrier between the body and the external world.

The barrier function of the stratum corneum with respect to moisture is considerable. Since the cells of the human body are mostly aqueous, the body in total is mostly water. This water could evaporate if not for the barrier function of the stratum corneum which holds the water in, much like a water balloon holding water.

Since a wound is a break in the skin, it will allow the escape of moisture vapor from the underlying moist tissues and the eventual death of superficial cells—called dehydration necrosis—which contributes to the formation of a scab along with the drying of released blood and sera. The scab itself presents very little barrier to further moisture loss.

The principals discussed in U.S. Pharmacist reference, Moist Wound Healing, infection control during wound care is an important consideration. There exists an ongoing controversy as to the methods used to prevent or treat local infection of wound tissues. One of the main concerns about moist local wound environment which may facilitate healing speed is that it also may facilitate infection rates. The rationale behind the concern is that a moist, warm environment is not only ideal for the growth and movement of tissue cells, but it is also conducive to bacterial proliferation. However, this theory have been disproved.

II. Over the Counter Medicines and the Food and Drug Administration

According to the Center for Drug Evaluation and Research (CDER), the United States Food and Drug Administration (FDA) determines whether medicines are prescription, meaning medicines that are safe and effective when used under a doctor's care, or nonprescription, meaning medicines that are safe and effective for use without a doctor's care. As such, the FDA has the authority to decide when a prescription drug is safe enough to be sold directly to consumers as over-the-counter medicines (OTCs).

OTCs have become increasingly popular over the past three decades and have allowed the population to take a more proactive and “hands on” approach toward health care. In fact, more than 700 products presently sold over-the-counter use ingredients or dosage strengths that were once available only by prescription 30 years ago. As the CDER states, increased access to OTCs is especially important for our maturing population. Two out of three older Americans rate their health as excellent to good, while four out of five report being affected by at least one chronic illness. Thus, as the life expectancy of humans continues to increase, these OTCs have become especially important to battle common ailments such as bacterial skin infections, acne, psoriasis, fungal infections, allergic reactions and the like.

Currently, many topical OTC medicines are available to the general public to help treat the afore referenced ailments. Some of the major advantages of using these OTCs topically include: the ease of availability, the avoidance of risks and inconvenience of parenteral treatment, the avoidance of the variable absorption and metabolism associated with oral treatments, continuity of drug administration, permitting use of pharmacologically active agents with short biological half-lives, potential reduction of gastrointestinal irritation in systemic administration and treatment of cutaneous manifestations of disease usually treated systemically. However, a limitation to topical OTCs is that they are restricted in effectiveness due to the impermeability of skin.

III. The Skin Barrier

The skin acts as a barrier to an ingress of pathogens and toxic chemicals and an egress of physiologic fluids. This impermeability is the result of normal physiologic changes in developing skin.

A typical cell in the epidermis is formed in the basal layer and takes approximately thirty days for the cell to migrate from the basal layer of the epidermis to the outer layers of the stratum corneum, where the cells are sloughed off and discarded. As the cell migrates outward from the basal layer, it progressively keratinizes, that is, becomes impregnated with the fibrous protein keratin, until it reaches the relatively impermeable outer layer, the stratum corneum.

The stratum corneum is an extremely thin surface layer with substantial barrier properties. The envelopes of the cells in the stratum corneum tend to be mainly polar lipids, such as ceramides, sterols and fatty acids, while the cytoplasm of the stratum corneum cells remains polar and aqueous. Despite the close packing of the cells, some 15% of the stratum corneum is intercellular and generally lipid based. It is generally recognized that over the thirty-day cell migration span, short-term penetration of external substances occurs through the hair follicles and the sebaceous apparatus while long-term penetration occurs across cells. Thus, many topical OTC medicines, which were formerly prescription, have poor penetration across the epidermal lipid barrier, thereby frustrating attempts to deliver clinically significant doses via topical application.

I believe the antibacterial/anti-inflammatory compositions of the present invention increases the penetration across the epidermal lipid barrier, thereby delivering a higher concentration of the active ingredients around the damaged cells and significantly improving the treatment.

The antibacterial components of the present invention inherently protects the wound from possible bacterial proliferation due to multiple mechanisms of action attacking the invading bacterial cell simultaneously.

The anti-inflammatory components of the composition counter skin irritation and inflammation from the wound responses, as well as the irritation from a metabolite from one of the antibacterial components.

The compositions of this invention address the problem of skin dehydration by incorporating a moisturizing oil and skin nutrient derived from a natural food product. By continuously hydrating the wound for a moist environment and providing skin nutrients, this component aids the body in the protection and repair of damaged tissue.

IV. FDA Approved Ingredients

A. Benzoyl Peroxide Benzoyl peroxide (BZP) is a potent, non-toxic oxidant which has long been used for treatment of dermatological lesions. It is known to be an effective antimicrobial and anti-keratolytic agent useful in the treatment of various skin afflictions, notably acne and rosacea.

BZP is a potent bacteriostatic agent oxidizing P. acnes and causing a reduction in free fatty acids, thus decreasing the inflammatory response. It also acts as a mild comedolytic through its drying and desquamating properties. It is available both OTC and by prescription in various forms, such as creams, gels, lotions, liquids and soaps. All strengths have equal antibacterial activity, but higher concentrations are more comedolytic.

Less irritating liquid and cream preparations are preferred for patients with dry skin, while the addition of a moisturizer/skin nutrient, such as Emu Oil, is especially beneficial.

Due to the oxygen to oxygen (O—O) bond, BZP is very reactive and unstable when combined with other active ingredients. If compounded with other pharmaceutical preparations by a pharmacist or a manufacturer, the resulting product has a short shelf-life of approximately 30 days to 60 days. The method of use of the present invention solves this potential degradation problem.

BZP pharmacological action on the skin is a result of metabolizing BZP to hydrogen peroxide and benzoic acid, of which both have antibacterial properties. While hydrogen peroxide is nonirritating to the skin, benzoic acid may cause irritation. The anti-inflammatory component of the present invention composition counteracts the potential irritation from the benzoic acid.

Medline® Plus Information states that “[o]nce a medicine has been approved for marketing for a certain use, experience may show that it is also useful for other medical problems. Although these uses are not included in product labeling, benzoyl peroxide is used in certain patients with the following medical conditions: Decubital ulcer (bed sores) and Stasis ulcer (a certain type of ulcer). Other than the above information, there is no additional information relating to proper use, precautions, or side effects for these uses.”

It is believed that the skin irritation of the product BZP causes such discomfort that use is limited. The composition of the present invention solves the problem of this discomfort.

As described in U.S. Pat. No. 4,923,900 to De Villez, granted May 1990, BZP is a keratolytic and desquamative agent, which possesses a broad antibacterial activity. BZP is a potent, nontoxic oxidant which has long been used for treatment of dermatological lesions and known to be an effective antimicrobial and anti-keratolytic agent. Described is BZP's chemical instability and skin irritation from metabolizing to benzoic acid. Glycerol is noted as being added to the preparations as a soothing emollient in an effort to counter skin irritation. Thus, Emu oil can replace glycerol as an emollient.

The method of use of this invention, in conjunction with Emu Oil and the anti-inflammatory components of these formulations, solves these potential problems by reducing or eliminating the skin irritation and allergic reaction.

Many prescription combinations of BZP are combined with antibiotics, such as erythromycin, a topical antibiotic, as described in U.S. Pat. No. 4,497,794. Compositions prepared generally as described in the '794 patent are sold under the trademark Benzamycin® by Dermik Laboratories, as a widely prescribed treatment for acne and is recognized in some cases to be more effective than treatment with either erythromycin or BZP alone. The components described in the '794 patent are combined prior to dispensing by a pharmacist. However, the method of use of the present invention simplifies the dispensing by the pharmacist and increases useable shelf life of BZP pharmaceuticals for the consumer.

U.S. Pat. No. 6,117,843 issued to Baroody, et. al. in September 2000 discloses a pharmaceutical composition containing clindamycin and BZP for the treatment of acne. The composition is stable for several months when stored at room temperature. Methods of preparing and of using the composition are disclosed.

U.S. Pat. No. 6,262,117 issued to Sefton in July 2001 discloses an invention that provides a method for treating acne vulgaris by serially applying a topical composition of azelaic acid and a topical composition of BZP.

B. Hydrocortisone

The most widely prescribed drugs to treat dermatologic disease of acute and chronic inflammatory dermatoses are corticosteroids such as hydrocortisone. Since the introduction of corticosteroids in the early 1950s for dermatologic diseases, topical corticosteroid therapy continues to be the mainstay for the management of a broad spectrum of inflammatory dermatoses. In fact, approximately 50% of prescriptions written by dermatologists are for topical corticosteroids. Although systemic corticosteroids are often required in some severe dermatologic diseases, topical treatment is preferred in most responsive cases because it causes fewer side effects.

Chemical mediators such as prostaglandins, kinins, histamine and liposomal enzymes increase the inflammatory response in humans by causing pain, increased vascular permeability and vasodilation (the opening up of the capillaries of the skin in response to warm temperatures, thus increasing the flow of blood to the surface of the body). Hydrocortisone is a topical corticosteroid in which part of its activity is hypothesized to be due to the drug binding to steroid receptors within the cell, forming a complex, entering the nucleus and binding to DNA. This binding modifies mRNA and alters subsequent protein synthesis of the chemical mediators. Corticosteroids also induce phospholipase A2 inhibitory proteins (lipocortins) that decrease the formation of these mediators. Finally, the migration of macrophages and leukocytes is decreased through the reversal of vascular dilation and permeability, thus decreasing the severity of the inflammation.

Currently, the only mild steroid easily accessed by the consumer is hydrocortisone in either 0.5% or 1% concentration. Since there is no correlation between the quantity of hydrocortisone applied and the degree of penetration, consumers should not apply too much of this steroid to his or her skin. It should be noted, however, that systemic side effects from nonprescription hydrocortisone are rare. Even if the entire contents of a 30-gram tube of 1% hydrocortisone were absorbed, the amount would total not more than 300 milligrams, which is only slightly higher than the approved initial oral dose of 240 milligrams/dose, although uncontrolled continued exposure to higher concentrated potent corticosteroids may increase the likelihood for systemic side effects.

Although some dermatoses may require therapy with a potent corticosteroid initially, treatment with hydrocortisone is often sufficient and is less likely to cause adverse reactions. Although fluorinated corticosteroids are generally potent and efficacious, fluorination is not essential for increase anti-inflammatory potency. Potent corticosteroids are customarily used for severe or resistant dermatoses such as psoriasis and chronic neurodermatitis.

Prior to the present invention, dermatoses such as discoid lupus erythematosus, lichen planus, granuloma annulare and psoriasis of the palms, soles, elbows and knees or psoriatic plaques usually require potent corticosteroids, which are by prescription only.

Local side effects have become more frequent with the clinical use of newer and more potent glucocorticosteroidal analogs. The most common adverse reaction is skin atrophy, i.e., a thinning of the epidermis and dermis accompanied by telangiectasia and striae (permanent scars). Minimal trauma to atrophic skin may also produce purpuric lesions.

The nutrient moisturizing component of the present invention aids in the thickening of the skin multiple times, thereby neutralizing the potential adverse reaction of the hydrocortisone skin atrophy. This component also aids in the increased penetration and concentration of the active ingredients, decreasing the need for more potent corticosteroids.

U.S. Pat. No. 5,998,395, issued to Kligman in December 1999, describes methods of treating inflammatory dermatosis. A comparative potency list of various corticosteroid preparations is documented therein. Therapeutic efficacy of steroid therapy can often be enhanced by increasing the potency of the steroid or by using special enhancers, such as occlusive dressings.

C. Emu Oil

Approved for human use by the FDA in July 1991, Emu Oil is a natural food product that is a combination of various natural fatty acids. The oil comes from a subcutaneous layer of fat found on the back of the Emu bird. The Emu bird, Dromiceius novaehollandiae, is located in Australia and is a large, flightless bird which resembles an ostrich. Aborigines of Australia have used this oil for hundreds of years for medicinal purposes. It is a very effective skin nutrient, moisturizer, emollient and emulsifier that aids in healthy skin integrity.

The oil is edible and is a non-irritant. It also does not irritate the mucous membranes of the eyes. At room temperature, Emu Oil is non-irritant to most skin types and does not present an inhalation hazard. Emu Oil, being a fine oil, also possesses a high degree of emollient/emulsification properties, thus having good “blendability.” In practice, this means that Emu Oil has the ability to blend, or make oil and water miscible, producing a cream that does not feel oily to the touch. Researchers believe that Emu Oil's unique combination of saturated and unsaturated fatty acids may explain its ability to enhance the willingness of the upper layers of the skin to retain water. For example, application of Emu Oil has been demonstrated to increase the overall thickness of the human skin by approximately 2½ to 5 times, thus reducing its tendency to form wrinkles. Thus, Emu Oil, which is actually non-comedogenic, has been used in the cosmetics industry. Additionally, Emu Oil has been reported to have antibacterial and anti-inflammatory properties.

While I believe the following theories, I do not wish to be bound by them: (1) that Emu Oil exhibits suitable transdermal penetrating characteristics whereby when the oil is combined with other active ingredients, penetration of the composition as a whole results in increased pharmaceutical concentrations within and around the cellular area of the chosen site; (2) increased pharmaceutical concentration penetration is a result of Emu Oil's non-phosphorus composition; (3) that by increasing the transdermal absorption amount of the active ingredients with the addition of Emu Oil, a significantly elevated synergistic pharmacological response, which may be catalytic, is achieved; (4) that Emu Oil is a natural product that directly feeds nutrients to the cells around the wound and moisturizes the wound area for speed of healing; (5) that Emu Oil increases the absorption of the active ingredients of hydrocortisone and BZP as a whole four to five times than without the addition of Emu Oil; (6) Emu Oil has anti-inflammatory and antibacterial properties that add more synergism to the whole and (7) the multiple different mechanisms of action of all these ingredients work harmoniously, attacking the skin infections in an unexpected efficacious manner, resulting in a highly effective antibacterial/anti-inflammatory skin infection treatment.

Various patents discuss the use of Emu oil, for example, U.S. Pat. No. 5,662,921 issued to Fein et al. in September 1997 discusses therapeutic oral uses for Emu Oil to lower cholesterol and triglycerides. Emu Oil can be used topically to prevent scarring when applied to a newly received cut or burn. Additionally, it is known that Emu Oil also diminishes old scars, even stretch marks.

U.S. Pat. No. 5,958,384 issued to Holick in September 1998 teaches the topical or parenteral administration of Emu Oil to a mammal stimulates the proliferation of skin, as well as rejuvenating photo-damaged skin. In addition, it teaches that Emu Oil stimulates melannogenesis in the skin and can be used to treat disorders such as hypo-pigmentation.

Emu oil is considered by the FDA to be safe for human usage. U.S. Pat. No. 5,472,713 issued to Fein et al. in December 1995 gives extensive description of the material safety and therapeutic uses of Emu Oil.

As described in U.S. Pat. No. 6,531,126 issued to Farmer in March 2003, Emu Oil also possesses a high degree of emollient/emulsification properties, and hence has good “blendability”.

Researchers believe that its unique combination of saturated and unsaturated fatty acids may be an explanation for its ability to enhance the willingness of the upper layers of the skin to retain water. For example, application of Emu Oil has been demonstrated to increase the overall thickness of human skin by approximately 2.5-times, thus reducing its tendency to form “wrinkles”.

U.S. Pat. No. 5,849,334 issued to Rivlin in December 1998 teaches compositions comprising local anesthetic and Emu oil. The compositions are useful in a method of anesthetizing cornified skin comprising topically administering the subject compositions.

U.S. Pat. No. 6,303,132 issued to Nelson in October 2001 describes a topical progesterone composition containing a concentration of progesterone and a concentration of Emu Oil. The Emu Oil theoretically acts as a transdermal vehicle to quickly and efficiently carry the progesterone into the body.

V. The Threat of Drug Resistance

It is theorized that combining the above FDA approved ingredients, that is, benzoyl peroxide having antibacterial properties and Emu Oil having antibacterial properties, leads to a remarkable result wherein the bacterium of the skin affliction is attacked by three different antibacterial mechanisms of action: 1) oxygen from hydrogen peroxide, a metabolite of benzoyl peroxide, first reaching the bacteria, 2) the benzoic acid from the benzoyl peroxide breaking down attacking the bacteria and finally 3) the antibacterial properties of the Emu Oil attacking the bacteria. This multi-line action mechanism is especially important due to drug (or active ingredient) resistance by bacteria.

Because of the massive quantities of antibiotics being prepared and used, an increasing number of diseases are resisting treatment due to the spread of drug resistance. Much of the difficulty arises from drug misuse and drugs being frequently overused in the past. Often a bacterial pathogen is drug resistant because it has a plasmid bearing one or more resistance genes, such as plasmids called R plasmids. Once a bacterial cell possesses an R plasmid, the plasmid may be transferred to other cells quite rapidly through normal gene exchange processes, such as conjugation, transduction and transformation. Because a single plasmid may carry genes for resistance to several drugs, a pathogen population can become resistant to several antibiotics simultaneously, even though the infected consumer is being treated with only one drug.

It is known, however, that several methods can be utilized to discourage the emergence of drug resistance, including 1) administering the drug in a high enough concentration to destroy susceptible bacteria and most spontaneous mutants that may arise during treatment and 2) administering two different drugs simultaneously with the hope that each drug will prevent the emergence of resistance to the other. Thus, it is thought that by combining the FDA approved ingredients, benzoyl peroxide having antibacterial properties and Emu Oil, which is thought to have both antibacterial and anti-inflammatory properties, drug resistance will not be achieved because the antibacterial properties of benzoyl peroxide and Emu Oil will reach the bacteria at a large enough concentration to kill the bacteria and most spontaneous mutants that may arise during treatment or, in the alternative, that administering these two synergistically different products simultaneously will prevent the emergence of resistance to the other.

VI. OTCS and the Pharmacist

Even though many topical OTCs with the active ingredients of hydrocortisone or benzoyl peroxide can be found at gas stations, grocery stores, airport lobbies, beauty shops, diners, convenience stores and even hotel vending machines, the FDA does not require human interaction in the sale of OTCs with the exception of enforcing an age limit on the sale of nicotine products.

The result of this invention is a highly efficacious healthcare tool for the treatment of multiple skin infections. Like any precision tool, it is most effective and efficient when placed in the hands of a professional who understands the benefits that can be derived from applying the tool in a precise manner. Traditionally, the licensed physician has been the gatekeeper for access to the utilization of such a tool.

The expertise of the pharmacist is invaluable and often underutilized. As a result of trust and availability, the pharmacist is one of the first healthcare professionals a consumer may seek advice from when treating an ailment. Pharmacists are available everyday of the week, sometimes twenty-four hours per day.

One of the objectives of the present invention is the introduction of a third class of pharmaceuticals, similar to prescriptions that are OTC, sold only in pharmacies under the control of a pharmacist whom will now also be a gatekeeper. The pharmacist is the gatekeeper that can recommend the proper usage of this valuable healthcare tool being placed in his or her hands and possesses the knowledge to direct the consumer to proper uses and precautions. The pharmacists, as a gatekeeper, can direct the consumer to the proper healthcare professional for more difficult ailments.

VII. The Federal Food, Drug and Cosmetic Act of 1938

The federal Food, Drug and Cosmetic Act of 1938 (FDCA), 21 U.S.C. §§ 301-397 regulates drug manufacturing, marketing and distribution. Section 505(a) of the FDCA provides that “no person shall introduce or deliver for introduction into interstate commerce any new drug, unless an approval of an application file with the Food and Drug Administration . . . is effective with respect to such drug.” The term ‘new drug’ is defined by § 201(p)(1) of the FDCA, 52 Stat. 1041, as amended, 76 Stat. 781, as “any drug . . . not generally recognized among experts qualified by scientific training and experience to evaluate the safety and effectiveness of drugs as safe and effective for use under the conditions prescribed, recommended or suggested in the labeling thereof.”

Thus, because the all the components of the present invention are designated as OTCs by the FDA, the composition and method of the present invention does not violate the FDCA.

VIII. Method of Delivery

The active component benzoyl peroxide has a well-known instability problem when combined with other active ingredients. BZP metabolizes to hydrogen peroxide with a double oxygen bond and benzoic acid. Stability of BZP is approximately 30 days to 60 days when combined with other active components. This instability results in a short shelf life and manufacturing problems, thereby creating the need for a more desirable and available consumer product.

The preferred vehicle for delivery of the present composition is a pharmaceutical delivery syringe for pairing synergistic active ingredients that is composed of three pieces: a barrel, a piston and a commonly shared split nozzle. The barrel has two syringe compartments that are attached to one another and are connected to the split nozzle. The piston has two plungers which are bridged together. When a user applies force to the piston, the substances in the syringe compartments are delivered through the respective side of the split nozzle onto a user's skin, whereby the consumer disperses and combines the components of the composition upon the skin.

After contiguous delivery of the isolated components on the afflicted area, the remaining contents must be protected from contamination from the environment, as well as cross-contamination from the individual components. Thus, a clean-out nozzle has been specifically designed to provide a means for the consumer to protect the pharmaceutical contents.

The present invention is an antibacterial/anti-inflammatory composition and method of pairing two synergistic active ingredients of current OTC pharmaceuticals in conjunction with an enhancing agent for increased penetration of the components as a whole, resulting in a highly absorbed and efficacious skin treatment. Without the method of use of the present invention, these paired pharmaceuticals would not only decompose in a few weeks, but would be designated as prescription only as defined by the FDCA.

As previously stated, the individual components of the present invention are designated as OTCs by the FDA. If the individual active ingredients are combined into one composition and introduced into interstate commerce as such, they are classified as prescription-only and are covered under the FDCA. However, with the method of use of the present invention, individual components are isolated from one another and remain classified as OTCs.

By providing such a the method of use, the components become contiguously delivered beyond the plane of the physical delivery system. The consumer, or his or her agent, admixes components on the skin whereby the resultant composition does not violate the FDCA.

The concept and method of use of these pharmaceuticals whereas a third class of topical pharmaceuticals are created opens up a unique avenue for the utilization of a pharmacist's expertise and at the same time provide a very economical, highly effective service, readily available product to the consumer. Therefore, this valuable healthcare tool will be sold only through pharmaceutical wholesalers to pharmacies, hospitals or other healthcare facilities whereby advice and guidance from trained healthcare professionals, namely pharmacists, may be given for the selection of the best topical OTC product and treatment available to the consumer.

Numerous patents describe various types of delivery systems. For instance, Spanish Pat. No. 8803996 issued to Marsal Tallo in July 1990 is a single tube with twin compartments within. The exits are adjacent to one another in the one delivery nozzle. Squeezing each side of the twin compartments results in a contiguous dispensing of two products. Although not shown, a cap must be apply over nozzle, with twin orifices, whereas cross-contamination takes place within interior of cap after first use. After squeezing each side of tube to deliver products, it is unknown whether equal pressure is being applied. Precision measurement of equal quantity of products are difficult.

In addition, U.S. Pat. No. 4,121,739 issued to Devaney et al. in October 1978 teaches a dispenser with unitary plunger and sealed construction commonly used to dispense an epoxy glue adhesive. The dispenser delivers precisely metering viscous fluids from a cartridge. However, the volume of the compartments and the protective closures are insufficient.

Next, U.S. Pat. No. 5,174,475 issued to Day et al. in December 1992 is a sequential dosing of antifungal and anti-inflammatory components of prescription only nature. The cumbersome apparatus varies dosages from a mixture of the components, dispensing only one of the components after a time period of treatment. Another apparatus taught in the '475 patent is a device wherein two tubes are fastened end to end with varying quantities of pharmaceuticals to be delivered at various sequential times.

Next, U.S. Pat. No. 5,269,441 issued to O'Meara in December 1993 is a dual chamber container assembly having two adjacent compartments separated by a pleated common wall segment. The components exit from two points where they are mixed externally. The problem with this type of device, however, is that the quantity of the components coming from either tube is not metered or measurable. The finger pressure can vary on either tube which can result in unknown ratio quantity being delivered.

In U.S. Pat. No. 5,324,505 issued to Kornettka et al. in June 1994, a striped, multicolored toothpaste and dispenser are taught that utilize strands for separation of components. The dispenser is difficult to use because of the new formulations viscosities being low and all the components will be delivered intermingled, violating the principles of separation of dissimilar products this patent is founded upon. In addition, the intermingling may propel products into a prescription-only status as well as degradation.

U.S. Pat. No. 5,310,091 issued to Dunning et al. in May 1994 teaches a dual product dispenser for simultaneously dispensing and mixing a pair of fluid products, such as chemically reactive resins, from a pair of axial adjacent front and rear chambers. A piston is mounted within each of the chambers and is moveable with respect to the hollow interior of the respective chamber for dispensing the fluid product. This particular patent uses a caulking gun type of delivery system that is bulky for pharmaceutical delivery.

With respect to U.S. Pat. No. 5,290,259, issued to Fisher in March 1994, a double syringe delivery system designed specifically for the dental arts to deliver liquids to chosen sites within the mouth is taught. The syringes are clipped together and long small tubes deliver components to chosen site where the very low viscosity liquid forms intermingle. This method of delivery would be very difficult for higher viscosity bases, such as creams and gels.

U.S. Pat. No. 5,720,416 issued to Izoe in February 1998 teaches a dispenser for viscous liquids in which a hollow, cylindrical body is provided with stationary walls. A rotatable member with vanes is situated within the body so that rotation of the vanes will cause the liquids contained within the body to be squeezed out through a nozzle in one end of the main body. The other end of the main body is provided with a rotatable cap to turn the vans within the body. However, this mechanism may intermingle the products violating the principles this patent is founded upon.

Finally, U.S. Pat. No. 6,448,233 issued to Lefevre et al. in September 2002 describes a BZP two-pump dispenser system in order to keep components separate before an admixture on the skin area of use. Higher concentrations are used in separate compartments as described whereas the mixtures of base vehicles decrease active ingredient concentrations by approximately 50%.

The present invention is a pairing of an over-the-counter (OTC) antibacterial benzoyl peroxide 10% and an OTC mild steroid, hydrocortisone 1%, with a natural food product, Emu Oil. The addition of Emu oil theoretically increases transdermal penetration of the active components four to five times the normal penetration rate than when applied individually without Emu Oil.

Emu oil has been reported to have multiple benefits for the skin, such as acting as an anti-inflammatory agent, acting as an antibacterial agent, providing skin nutrients, acting as an emollient, lubricant and moisturizer, as well as a carrier for transdermal penetration of active components of pharmaceuticals. Emu oil, having excellent blendability, can easily replace mineral oil or other bland oils used as vehicle bases in skin formulations.

With a preferred delivery syringe, which isolates the active components, the OTC embodiment of the pairing of Benzoyl Peroxide/Hydrocortisone/Emu Oil can comprise concentrations of benzoyl peroxide from 1% to 10%, hydrocortisone from 0.1% to 1% and Emu oil from 3% to 99%, with the preferred concentrations being benzoyl peroxide 10%, hydrocortisone 1% and Emu oil from 3% v/v to 15% v/v. A minimum of 3% v/v Emu oil is required for optimum transdermal penetration of the active components as a whole.

Another embodiment of the composition of the present invention also utilizes the preferred delivery syringe to isolate the active components, creating a prescription-only embodiment of pairing of Benzoyl Peroxide/Hydrocortisone/Emu Oil wherein the benzoyl peroxide concentration is between 5% to 30%, the hydrocortisone concentration is between 1% to 5% and the Emu Oil concentration is between 3% to 99%. However, the preferred concentrations for the prescription-only embodiment is benzoyl peroxide 20%, hydrocortisone 2.5% and Emu oil between 3% to 15%. v/v.

Another prescription-only embodiment which also utilizes the preferred delivery syringe to isolates the active components, is the pairing of Benzoyl Peroxide with selected low-potency steroid and Emu Oil wherein the concentration for benzoyl peroxide is between 5% to 30% and the concentration for Emu Oil is between 3% to 99%. In addition, these concentrations are paired with a low potency steroid selected from the group consisting of cortisone, prednisone acetate, prednisone valerate, prednisolone, betametasone dipropionate, fluocinolone acetonide, dexamethasone, methylprednisolone, and desonide. However, the preferred embodiment of this prescription-only composition is benzoyl peroxide 20%, betamethasone 0.05% and Emu oil from 3% to 15% v/v.

The present invention provides a convenient solution to short shelf life and the products remaining as OTC pharmaceuticals by utilizing a delivery syringe to enable separate containment of individual compositions, i.e., a first composition comprising benzoyl peroxide 10% and a second composition comprising hydrocortisone 1% with a transdermal penetration enhancer, Emu Oil of 3% to 15% v/v, added to either formulation. According to the present invention, the benzoyl peroxide and hydrocortisone with Emu Oil generate a resulting composition when mixed upon delivery, with the resulting composition being effective to apply the active ingredients benzoyl peroxide and hydrocortisone in an effective form. Finally, the admixing of the individual compositions, benzoyl peroxide and hydrocortisone with Emu Oil, takes place on the skin at site of affliction by the consumer or their agent.

The present invention further discloses a method to produce an effective composition comprising benzoyl peroxide 10% and a second active ingredient, hydrocortisone 1% with Emu oil minimum 3% v/v, by on site admixing of two individual compositions, one or each containing Emu oil, each comprising a predetermined ratio amount of benzoyl peroxide and hydrocortisone. The method of use of the delivery syringe according to the invention enables the preparation and use of separate compositions, on the one hand a composition comprising benzoyl peroxide 10% as the active ingredient with Emu oil and on the other hand a composition comprising hydrocortisone 1% and Emu oil. These separate compositions are prepared in such a way that the individual requirements for stable formulation of each active ingredient are fulfilled. In addition, the separate compositions are prepared in such a way that the final composition is effective to deliver the active ingredients present in each individual composition in a form suitable and effective for direct application.

The preferred ratio in which the two individual compositions are delivered by the delivery system is 1:1. The dispersion of the components on the skin site results in 50% concentrations from the isolated compartments of the delivery system. On site, after dispersion, benzoyl peroxide is 5% and hydrocortisone is 0.5%, with the combining of the vehicle bases. Normally, active ingredient penetration is 1% or variable thereof through the dermis. Emu oil of at least 3% v/v acting as a transdermal transporter increases dermal penetration of the active ingredients from 4 to 5 times. Thus, the resulting pharmacological response from increased absorption is equivalent to the application of benzoyl peroxide 20% to 25% and hydrocortisone 2% to 2.5%. Additionally, penetration of the active ingredients is deeper into the tissue below the dermis for increased therapeutic response toward the wound damage being treated.

Emu oil is thought to have antibacterial properties. Benzoyl peroxide metabolizes to hydrogen peroxide and benzoic acid, both of which are antibacterial, and in conjunction with Emu Oil antibacterial activity, the composition attacks bacteria with three different mechanisms of action.

Because Emu Oil is thought to have anti-inflammatory properties, further synergistic activity is created when combining the active ingredients benzoyl peroxide, hydrocortisone and Emu Oil. Hydrocortisone and Emu Oil work synergistically to counteract the skin irritation from one of the metabolite of benzoyl peroxide, benzoic acid.

Further synergistic activity is the moisturizing, skin nutrient natural ability of Emu Oil, which soothes and feeds the surrounding damaged tissue, keeping the tissue moist for acceleration of healing.

By pairing these OTC medicines together using a pharmaceutical delivery syringe, a more effective antibacterial/anti-inflammatory treatment for multiple skin conditions is contiguously delivered on site of affliction, while isolated components remain classified as OTC pharmaceuticals.

The use of the antibacterial/anti-inflammatory composition comprising the pairing of two synergistic active ingredients with Emu Oil will allow for a deeper penetrating, more effective skin treatment for acne and rosacea, skin infections and wound healing among others.

The prior art includes the following patents:

| Patent No. | | | |
|---|---|---|---|
| (U.S. unless | | | Issue/Publication |
| otherwise noted) | Inventor | Filing Date | Date |
| 2002/0146440 A1 | Smith | Apr. 09, 2001 | Oct. 10, 2002 |
| Spain 8803996 | Tallo | Nov. 18, 1988 | Jul. 16, 1990 |
| 4,121,739 | Devaney et al. | Apr. 20, 1977 | Oct. 24, 1978 |
| 5,174,475 | Day et al. | Mar. 26, 1991 | Dec. 29, 1992 |
| 5,269,441 | O'Meara | Jan. 31, 1992 | Dec. 14, 1993 |
| 5,324,505 | Kornettka et al. | Jun. 28, 1990 | Jun. 28, 1994 |
| 5,310,091 | Dunning et al. | May 12, 1993 | May 10, 1994 |
| 5,290,259 | Fisher | Feb. 18, 1993 | Mar. 01, 1994 |
| 5,720,416 | Izoe | Aug. 14, 1996 | Feb. 24, 1998 |
| 6,448,233 | Lefevre et al. | Jul. 08, 1997 | Sep. 10, 2002 |
| 5,662,921 | Fein et al. | Jun. 07, 1995 | Sep. 02, 1997 |
| 5,958,384 | Holick | Apr. 27, 1998 | Sep. 28, 1998 |
| 5,472,713 | Fein et al. | Nov. 23, 1994 | Dec. 05, 1995 |
| 6,531,126 | Farmer | May 07, 2001 | Mar. 11, 2003 |
| 5,849,334 | Rivlin | Sep. 29, 1997 | Dec. 15, 1998 |
| 6,303,132 | Nelson | Jul. 16, 1999 | Oct. 16, 2001 |
| 2003/0031724 A1 | Orthoefer et al. | May 16, 2001 | Feb. 13, 2003 |
| 2001/0033838 A1 | Farmer | May 07, 2001 | Oct. 25, 2001 |
| 5,998,395 | Kligman | Sep. 10, 1993 | Dec. 07, 1999 |
| 4,923,900 | De Villez | May 13, 1986 | May 08, 1990 |
| 4,401,835 | Tarasov | Sep. 17, 1981 | Aug. 30, 1983 |
| 4,497,794 | Klein et al. | Jan. 03, 1983 | Feb. 05, 1985 |
| 6,262,117 B1 | Sefton | Feb. 18, 1999 | Jul. 17, 2001 |
| 6,117,843 | Baroody et al. | May 13, 1997 | Sep. 12, 2000 |

### SUMMARY OF THE INVENTION

The primary object of the present invention is to provide an antibacterial/anti-inflammatory composition for skin infections.

A further object of the present invention is to provide an antibacterial/anti-inflammatory composition for skin infections that is comprised of two synergistic dissimilar active ingredients that can be independently purchased without a prescription.

An even further object of the present invention is to provide an antibacterial/anti-inflammatory composition that has a long shelf life.

An even further object of the present invention is to provide an antibacterial/anti-inflammatory composition that is less-irritating than current acne and rosacea treatments.

A further object of the present invention is to provide a treatment that can be sold as an OTC product for ease of access for the consumer.

A further object of the present invention is to place into the hands of qualified health care professionals, notably pharmacists, a readily available healthcare tool for the healing of skin infections.

A further object of the present invention is to provide a vehicle for delivery of the antibacterial composition that prevents cross-contamination of separate pharmaceuticals contained therein.

An even further object of the present invention is to provide a vehicle for delivery of the antibacterial/anti-inflammatory composition that protects the active ingredients from intermingling prior to use.

An even further object of the present invention is to provide a vehicle for delivery of the antibacterial/anti-inflammatory composition that permits ingredients with higher viscosity bases to intermingle during use.

The present invention fulfills the above and other objects by providing a multipurpose composition comprising the pairing of two synergistic dissimilar active ingredients with Emu Oil, which acts as a transdermal penetrating transporter, for a more efficacious formulation for topical application treatments.

The two synergistic active ingredients are hydrocortisone 1% and benzoyl peroxide 10% which, when combined, have a short shelf life of about 30 to 60 days. After mixing the two ingredients, the benzoyl peroxide begins slow decomposition to benzoic acid and hydrogen peroxide. The double oxygen bond of benzoyl peroxide begins to disintegrate. After about 30 to 60 days, the double bond of the oxygen molecule of hydrogen peroxide is broken and ineffective, which results in a composition containing benzoic acid, more of a skin irritant, and hydrocortisone, resulting in a less effective product. Thus, although combining these ingredients manually is effective, the preferred method for delivering these ingredients to the skin is by using a vehicle that prevents the ingredients from mixing prior to use.

Additionally, although applying these two active ingredients onto the skin is effective, increased effectiveness of the delivery of the active ingredients occurs when combined with Emu Oil based on the theories that: 1) Emu Oil exhibits suitable transdermal penetrating characteristics whereby when the oil is combined with other active ingredients, penetration of the composition as a whole results in increased pharmaceutical concentrations within and around the cellular area of the chosen site, 2) the increased pharmaceutical concentration penetration is a result of Emu Oil's non-phosphorus composition and 3) by increasing the transdermal absorption amount of the active ingredients, with the addition of Emu Oil, a significant elevated synergistic pharmacological response, which may be catalytic, is achieved.

The preferred vehicle for delivery of the present composition is by using a pharmaceutical delivery syringe for pairing synergistic active ingredients that is composed of three pieces: a barrel, a piston and a commonly shared split nozzle. The barrel has two syringe compartments that are attached to one another and are connected to the split nozzle. The piston has two plungers which are bridged together. When a user applies force to the piston, the substances in the syringe compartments are dispersed through the respective side of the split nozzle onto the user's skin.

The addition of Emu Oil of at least 3% of composition is used as an emollient, moisturizer and lubricant to aid in the prevention of skin dryness. Additionally, Emu Oil theoretically increases the penetrability of the OTC active ingredients, hydrocortisone 1% and benzoyl peroxide 10%, through the skin barrier for increased concentration and activity. The combining of the ingredients on the afflicted skin by the consumer results in synergistic action of the antibacterial ability of benzoyl peroxide and Emu Oil, along with the anti-inflammatory ability of hydrocortisone and Emu Oil, in a more penetrating formulation for the treatment of skin afflictions.

Emu Oil has been reported to have antibacterial and anti-inflammatory properties. Thus, when the FDA approved active ingredient benzoyl peroxide having anti-bacterial properties is combined with FDA approved safe product, Emu Oil, a remarkable and complex result is thought to be achieved wherein the bacteria of the skin affliction are attacked by three different mechanisms of action: 1) the oxygen, a reducing agent from hydrogen peroxide, a metabolite of benzoyl peroxide, reaching the bacteria, 2) the benzoic acid from the benzoyl peroxide breaking down attacking the bacteria and finally 3) the antibacterial properties of the Emu Oil attacking the bacteria. This is especially important due to the threat of drug (active ingredient) resistance by the bacteria.

The above and other objects, features, and advantages of the present invention should become even more readily apparent to those skilled in the art upon a reading of the following detailed description in conjunction with the drawings wherein there is shown and described illustrative embodiments of the invention.

### BRIEF DESCRIPTION OF THE DRAWINGS

This invention is described by appended claims in relation to a description of a preferred embodiment with reference to the following drawings which are explained briefly as follows:

FIG. 1 is a side view of a first preferred vehicle for delivery of the present invention;

FIG. 2 is a perspective view of the embodiment of FIG. 1 with the clean-out nozzle placed on the split nozzle;

FIG. 3 is a perspective view of the barrel of the embodiment of FIG. 1;

FIG. 4 is a perspective view of the top of the barrel after cutting the nozzle seal of the embodiment of FIG. 3;

FIG. 5 is a perspective view of the piston of the embodiment of FIG. 1;

FIG. 6 is a cross sectional view along lines 6-6 of the barrel of the embodiment of FIG. 2;

FIG. 7 is a side view of a second preferred vehicle for delivery of the present invention;

FIG. 8 is a perspective view of the embodiment of FIG. 7; and

FIG. 9 is a cross sectional view along lines 9-9 of the barrel of the embodiment of FIG. 8.

### DESCRIPTION OF THE PREFERRED EMBODIMENT

Listed numerically below with reference to the drawings are terms used to describe features of this invention. These terms and numbers assigned to them designate the same features throughout this description.

```
  1. pharmaceutical delivery syringe
  2. first syringe compartment
  3. clean-out nozzle
  4. cap
  5. split nozzle
  6. plunger unit
  7. marker
  8. piston
  9. barrel
10. thread
11. first nozzle opening
12. bridge
13. second nozzle opening
14. second syringe compartment
15. nozzle seal
```

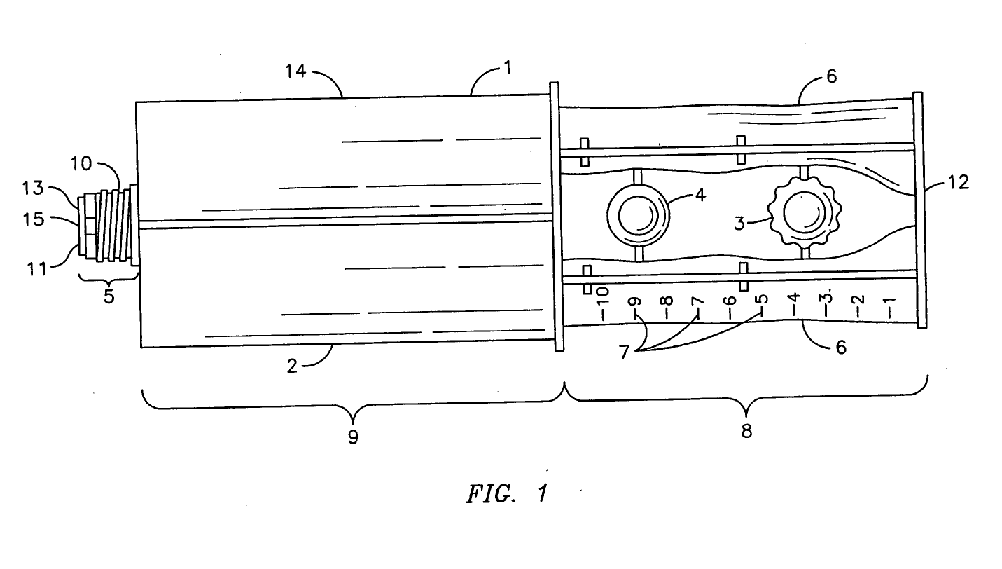

With reference to FIG. 1, a side view of a first preferred vehicle for delivery of the present invention is shown. The pharmaceutical delivery syringe 1 has a piston 8, a barrel 9 and a split nozzle 5. The piston 8 has two plunger units 6 connected via a bridge 12. One of the plunger units 6 has markers 7 to indicate the amount of pharmaceutical contents excreted. A clean-out nozzle 3 and a cap 4 are removably affixed to the open middle portion of the piston 8. The barrel 9 has a first syringe compartment 2 and a second syringe compartment 14 to house different over-the-counter (OTC) medicines. The syringe compartments 2 and 14 are connected to the split nozzle 5 so as the medicine in the first compartment 2 will exit through a first nozzle opening 11 while medicine in the second compartment 14 will exit simultaneously through a second nozzle opening 13.

To use the pharmaceutical delivery syringe 1, a user first separates the clean-out nozzle 3 and cap 4 from the piston 8. Then, the user cuts the nozzle seal 15 to expose a first and a second nozzle opening 11 and 13. Next, the user applies pressure to the bridge 12 of the piston 8 to make the pharmaceutical contents of each compartment 2 and 14 exit through the common split nozzle 5. Because the nozzle 5 is split, the pharmaceutical contents continue to be isolated until contiguously delivered on or near the site of the topical affliction to be treated. As pressure is applied to the bridge 12, precise quantities of pre-determined ratios of the dissimilar pharmaceuticals are extruded in close proximity to one another on or near the site of the affliction. As the dissimilar pharmaceuticals are extruded in a close adjacent path, there is a minute void region beyond the physical plane of the delivery syringe 1 whereas pharmaceuticals are not in contact. As each dissimilar pharmaceutical pass beyond the delivery syringe 1 and void region, they become contiguous on or near the site of the affliction. The user then simply disperses the dissimilar pharmaceuticals topically by using a finger in a circular motion to allow the pharmaceuticals to remain in the same area to cause a synergistic action.

The clean-out nozzle 3 is then placed over the split nozzle 5. The closure cap 4 may then be placed over the clean-out nozzle 3 to protect pharmaceuticals from drying and external contamination. The interior of the closure cap 4 may harbor cross-contamination of pharmaceuticals. Both the threaded clean-out nozzle 3 and cap 4 are provided for cleaning by the consumer, or their agent, of any cross-contamination accumulation.

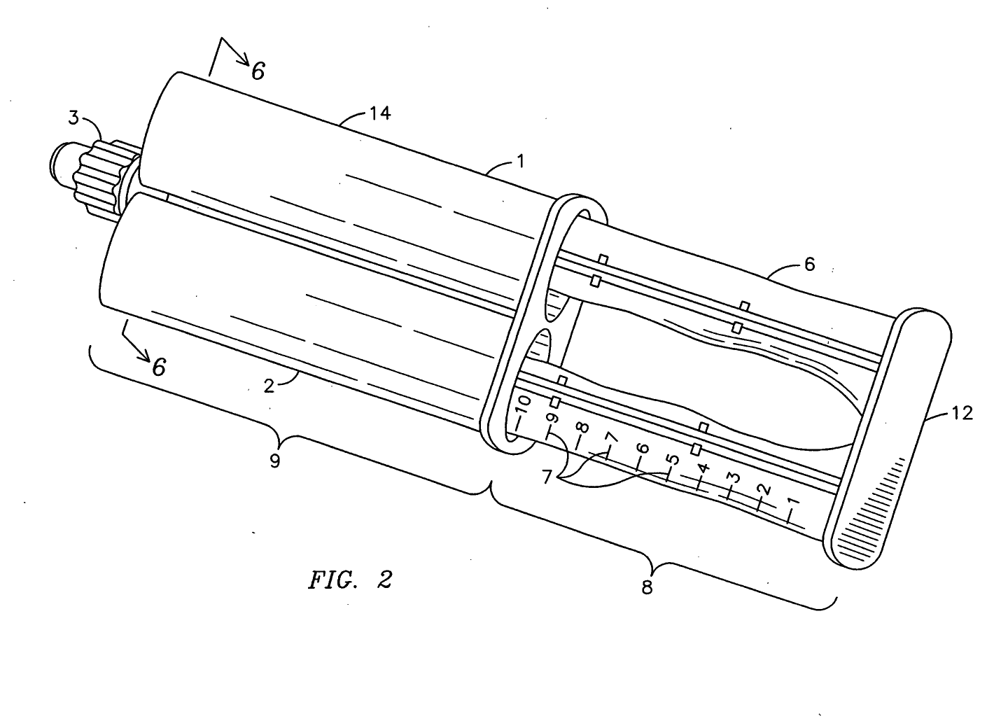

With reference to FIG. 2, a perspective view of the embodiment of FIG. 1 is shown with the clean-out nozzle 3 placed on the split nozzle 5. The clean-out nozzle 3 helps to prevent cross-contamination of the pharmaceuticals in the compartments 2 and 14.

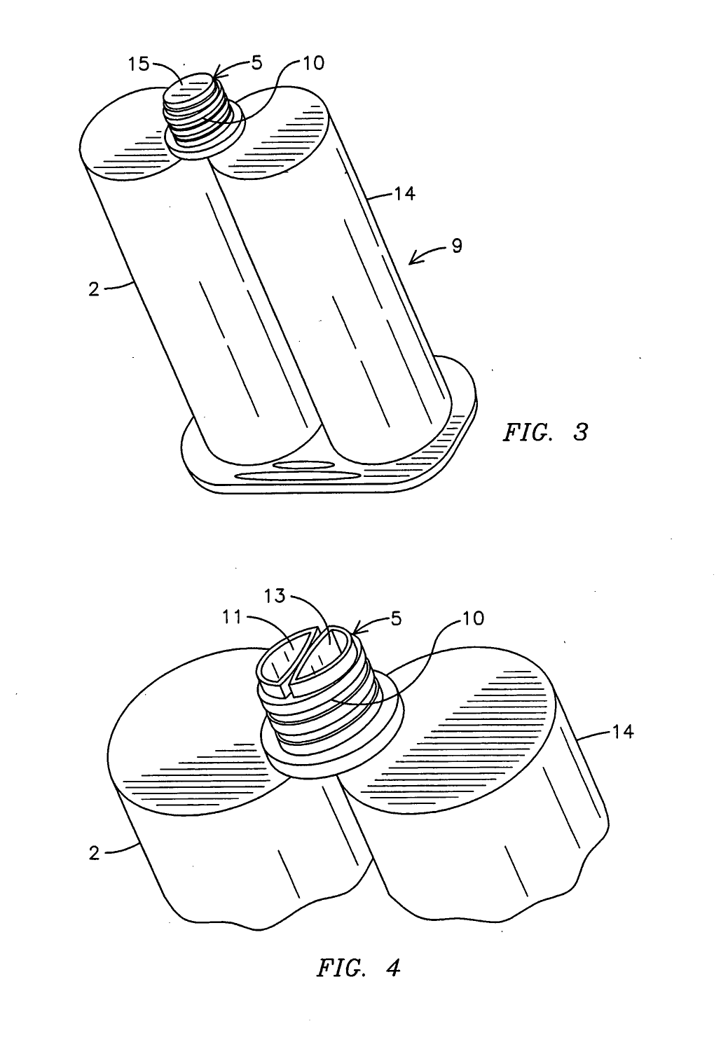

In FIG. 3, a perspective view of the barrel of the embodiment of FIG. 1 is shown. The split nozzle 5 has threads 10 to allow for the securing of a clean-out nozzle 3 or cap 4. A seal 15 is provided to keep the first and second nozzle openings 11 and 13 covered to keep the pharmaceuticals in their respective compartments 2 and 14 until the syringe 1 is ready for use.

With reference to FIG. 4, a perspective view of the top of the barrel after cutting the seal 15 of the embodiment of FIG. 3 is shown. The first opening 11 and second opening 14 have semi-circular openings for the allowance of pharmaceuticals to pass though.

With reference to FIG. 5, a perspective view of a piston 8 of the embodiment of FIG. 1 is shown. The piston 8 has two parallel plunger units 6 with each plunger unit 6 fitting into a syringe compartment 2 and 14. Markers 7 on the side of a plunger unit 6 indicate how much of the pharmaceutical is being dispersed.

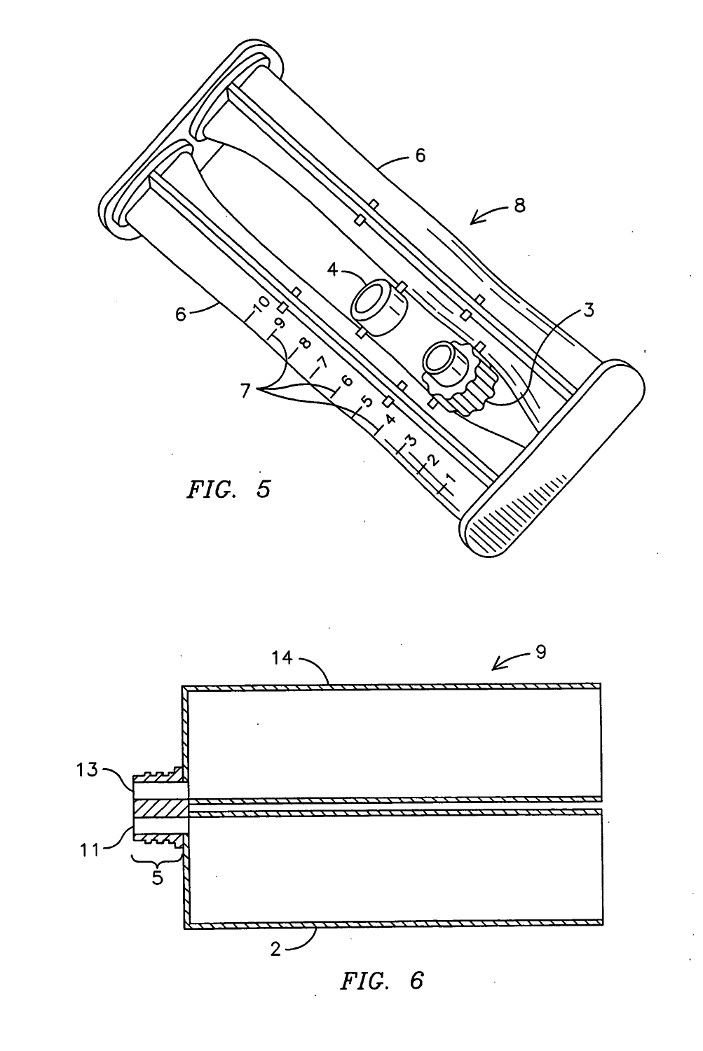

In FIG. 6, a cross sectional view along lines 6-6 of the barrel of the embodiment of FIG. 2 is shown. The pharmaceuticals located in the first syringe compartment 2 exit the split nozzle 5 via a first nozzle opening 11 while pharmaceuticals located in the second syringe compartment 14 exit the split nozzle 5 via a second nozzle opening 13. By having the pharmaceuticals exit the syringe 1 separately, synergistic actions will not occur until the moment the pharmaceuticals are at or near the site of the affliction and are rubbed into the skin by the user.

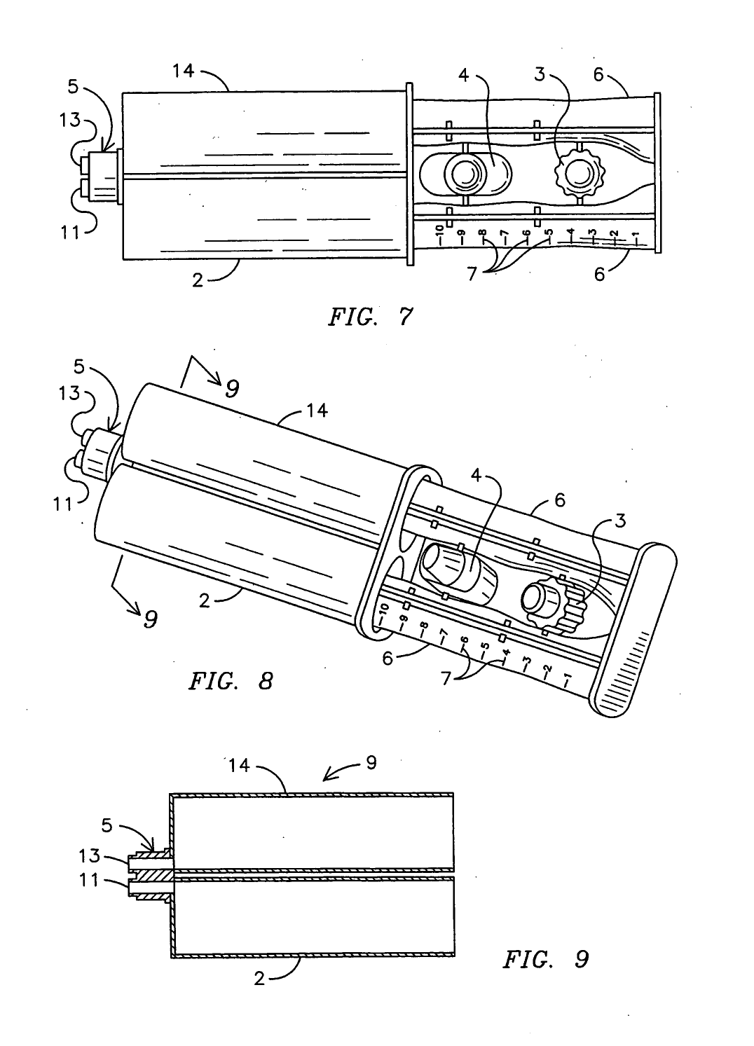

With reference to FIG. 7, a side view of a second preferred vehicle for delivery of the present invention is shown. The second preferred vehicle has the same elements listed and described in FIG. 1, however the split nozzle 5 and the cap 4 are shaped differently.

In FIG. 8, a perspective view of the embodiment of FIG. 7 is shown. The cap 4 is shaped so as to extend upward and away from the plunger unit.

Finally, with reference to FIG. 9, a cross sectional view along lines 9-9 of the barrel of the embodiment of FIG. 8 is shown. The first nozzle opening 11 and the second nozzle opening 13 are shaped so as to extend away from the barrel 9.

By pairing these over-the-counter medicines together using the pharmaceutical delivery syringe 1, a more effective antibacterial/anti-inflammatory treatment for multiple skin conditions is contiguously delivered on site of affliction, while in the preferred embodiment still remaining an over-the-counter pharmaceutical.

It is to be understood that while a certain form of the invention is illustrated, it is not to be limited to the specific form or arrangement of parts herein described and shown. It will be apparent to those skilled in the art that various changes may be made without departing from the scope of the invention and the invention is not be considered limited to what is shown and described in the specification and drawings.

Having thus described my invention, I claim:

1. A composition comprising:
hydrocortisone;
benzoyl peroxide; and
Emu Oil.

2. The composition of claim 1 wherein:
said hydrocortisone is at least 1%;
said benzoyl peroxide is at least 10%; and
said Emu Oil is at least 3%.

3. A pharmaceutical delivery syringe comprising:
a piston section having a predetermined size;
said piston section having at least two plunger units;
a barrel section sized slightly larger than said piston;
said barrel section having at least two syringe compartments sized slightly larger than said at least two plunger units;
a split nozzle attached to said barrel section;
a clean-out nozzle removably attached to said at least two plunger units; and
a cap removably attached to said at least two plunger units.

4. The pharmaceutical delivery syringe of claim 3 wherein:
said piston section has a first and a second plunger;
said barrel section has a first and a second syringe compartment;
said split nozzle has a first and a second opening;
said first plunger fits into said first syringe compartment;
said first syringe compartment opens to said first opening;
said second plunger fits into said second syringe compartment;
said second syringe compartment opens to said second opening; and
said first plunger has visible graduated markers.

5. The pharmaceutical delivery syringe of claim 3 wherein:
said at least two plunger units are equally sized and shaped; and
said at least two plunger units are parallel to each other.

6. The pharmaceutical delivery syringe of claim 3 wherein:
said at least two plunger units each have a top end;
said at least two plunger units have a bridge connecting said each top end to said each top end; and
at least one plunger unit has visible graduated markers.

7. The pharmaceutical delivery syringe of claim 3 wherein:
said at least two syringe compartments are equally sized and shaped; and
said at least two syringe compartments are parallel.

8. The pharmaceutical delivery syringe of claim 3 wherein:
said split nozzle has at least two openings;
said at least two openings open to said at least two syringe compartments; and
a seal is located over said at least two openings.

9. The pharmaceutical delivery syringe of claim 3 wherein:
said clean-out nozzle is threadably attachable to said split nozzle; and
said cap is threadably attachable to said split nozzle.

10. A method of applying a composition comprising hydrocortisone, benzoyl peroxide and Emu Oil, said method comprising the following steps:
a. applying a predetermined amount of hydrocortisone mixed with Emu Oil onto a predetermined skin area;
b. applying a predetermined amount of benzoyl peroxide near same predetermined skin area; and
c. massaging the hydrocortisone, benzoyl peroxide and Emu Oil into the skin.

11. A method of applying a composition comprising hydrocortisone, benzoyl peroxide and Emu Oil, said method comprising the following steps:
a. applying a predetermined amount of hydrocortisone onto a predetermined skin area;
b. applying a predetermined amount of benzoyl peroxide mixed with Emu Oil near same predetermined skin area; and
c. massaging the hydrocortisone, benzoyl peroxide and Emu Oil into the skin.

12. A method of applying a composition comprising hydrocortisone, benzoyl peroxide and Emu Oil, said method comprising the following steps:
a. applying a predetermined amount of hydrocortisone mixed with Emu Oil onto a predetermined skin area;
b. applying a predetermined amount of benzoyl peroxide mixed with Emu Oil near same predetermined skin area; and
c. massaging the hydrocortisone, benzoyl peroxide and Emu Oil into the skin.

13. The method of method claim 10 wherein:
said hydrocortisone is at least 1%;
said benzoyl peroxide is at least 10%; and
said Emu Oil is at least 3%.

14. The method of method claim 11 wherein:
said hydrocortisone is at least 1%;
said benzoyl peroxide is at least 10%; and
said Emu Oil is at least 3%.

15. The method of method claim 12 wherein:
said hydrocortisone is at least 1%;
said benzoyl peroxide is at least 10%; and
said Emu Oil is at least 3%.

16. A method of applying a composition comprising hydrocortisone, benzoyl peroxide and Emu Oil by using a pharmaceutical delivery syringe comprising a piston section having a predetermined size; said piston section having at least two plunger units; a barrel section sized slightly larger than said piston; said barrel section having at least two syringe compartments sized slightly larger than said at least two plunger units; a split nozzle attached to said barrel section; a clean-out nozzle removably attached to said at least two plunger units; and a cap removably attached to said at least two plunger units, said method comprising the steps of:
a. applying a downward force on said barrel section; and
b. using fingers to massage the hydrocortisone, benzoyl peroxide and Emu Oil into the skin.

17. A method for applying a composition comprising hydrocortisone, benzoyl peroxide and Emu Oil using a pharmaceutical delivery system comprising a piston section having a predetermined size; said piston section having at least two plunger units; a barrel section sized slightly larger than said piston; said barrel section having at least two syringe compartments sized slightly larger than said at least two plunger units; a split nozzle attached to said barrel section; a clean-out nozzle removably attached to said at least two plunger units; and a cap removably attached to said at least two plunger units, said method comprising the steps of:
a. inserting hydrocortisone and Emu Oil into the first syringe compartment;
b. inserting benzoyl peroxide into the second syringe compartment;
c. removing a seal from the split nozzle;
d. applying pressure to the piston section; and
e. rubbing the dispersed pharmaceuticals onto a surface.

18. A method for applying a composition comprising hydrocortisone, benzoyl peroxide and Emu Oil using a pharmaceutical delivery system comprising a piston section having a predetermined size; said piston section having at least two plunger units; a barrel section sized slightly larger than said piston; said barrel section having at least two syringe compartments sized slightly larger than said at least two plunger units; a split nozzle attached to said barrel section; a clean-out nozzle removably attached to said at least two plunger units; and a cap removably attached to said at least two plunger units, said method comprising the steps of:
a. inserting hydrocortisone into the first syringe compartment;
b. inserting benzoyl peroxide and Emu Oil into the second syringe compartment;
c. removing a seal from the split nozzle;
d. applying pressure to the piston section; and
e. rubbing the dispersed pharmaceuticals onto a surface.

19. A method for applying a composition comprising hydrocortisone, benzoyl peroxide and Emu Oil using a pharmaceutical delivery system comprising a piston section having a predetermined size; said piston section having at least two plunger units; a barrel section sized slightly larger than said piston; said barrel section having at least two syringe compartments sized slightly larger than said at least two plunger units; a split nozzle attached to said barrel section; a clean-out nozzle removably attached to said at least two plunger units; and a cap removably attached to said at least two plunger units, said method comprising the steps of:
a. inserting hydrocortisone and Emu Oil into the first syringe compartment;
b. inserting benzoyl peroxide and Emu Oil into the second syringe compartment;
c. removing a seal from the split nozzle;
d. applying pressure to the piston section; and
e. rubbing the dispersed pharmaceuticals onto a surface.

20. The method claim of claim 17 further comprising the step prior to d of:
detaching a clean-out nozzle and a cap from a piston section.

21. The method claim of claim 18 further comprising the step prior to d of:
Detaching a clean-out nozzle and a cap from a piston section.

22. The method claim of claim 19 further comprising the step prior to d of:
detaching a clean-out nozzle and a cap from a piston section.

23. The method claim of claim 17 further comprising the step prior to e of:
monitoring the amount of pharmaceuticals dispersed according to graduated markers on a plunger unit.

24. The method claim of claim 18 further comprising the step prior to e of:
monitoring the amount of pharmaceuticals dispersed according to graduated markers on a plunger unit.

25. The method claim of claim 19 further comprising the step prior to e of:
monitoring the amount of pharmaceuticals dispersed according to graduated markers on a plunger unit.

26. The method claim of claim 17 further comprising the step of:
placing the clean-out nozzle over a split nozzle.

27. The method claim of claim 18 further comprising the step of:
placing the clean-out nozzle over a split nozzle.

28. The method claim of claim 19 further comprising the step of:
placing the clean-out nozzle over a split nozzle.

29. The method claim of claim 17 further comprising the step of:
covering the clean-out nozzle with the cap.

30. The method claim of claim 18 further comprising the step of:
covering the clean-out nozzle with the cap.

31. The method claim of claim 19 further comprising the step of:
covering the clean-out nozzle with the cap.

32. The method claim of claim 17 wherein:
said hydrocortisone is 1%;
said benzoyl peroxide is 10%; and
said Emu Oil is at least 3%.

33. The method claim of claim 18 wherein:
said hydrocortisone is 1%;
said benzoyl peroxide is 10%; and
said Emu Oil is at least 3%.

34. The method claim of claim 19 wherein:
said hydrocortisone is 1%;
said benzoyl peroxide is 10%; and
said Emu Oil is at least 3%.
